(19) United States
(12) Patent Application Publication
(10) Pub. No.: US 2012/0245376 A1
(43) Pub. Date: Sep. 27, 2012

(54) METHOD FOR PRODUCING N-PROPYL ACETATE

(75) Inventors: Shigeru Hatanaka, Oita-shi (JP); Katsuyuki Tsuji, Kawasaki-shi (JP)

(73) Assignee: SHOWA DENKO K.K., Minato-ku, Tokyo (JP)

(21) Appl. No.: 13/513,920

(22) PCT Filed: Dec. 3, 2010
(86) PCT No.: PCT/JP2010/072184

§ 371 Date: Jun. 5, 2012

(30) Foreign Application Priority Data
Dec. 7, 2009 (JP) 2009-277491

(51) Int. Cl. C07C 67/283

(52) U.S. Cl. 560/265

(57) ABSTRACT

One object of the present invention is to provide a method for producing n-propyl acetate by the hydrogenation reaction with a hydrogenation catalyst, using an allyl acetate containing solution as a raw material, wherein the method can prevent the conversion rate of the substrate (allyl acetate) from decreasing with time and the product quality from deteriorating, and the present invention provides a method for producing n-propyl acetate including a first hydrogenation step in which a raw material solution containing allyl acetate and a hydrogen containing gas are reacted under a pressure P₁ of 1.0 MPa G (gage pressure) or more in the presence of a hydrogenation catalyst, to hydrogenate the allyl acetate to produce a hydrogenation reaction product containing n-propyl acetate: a gas-liquid separation step in which the hydrogenation reaction product is gas-liquid separated into to produce a crude n-propyl acetate solution containing n-propyl acetate: and a second hydrogenation step in which non-reacted allyl acetate contained in the crude n-propyl acetate solution is hydrogenated using hydrogen dissolved in the crude n-propyl acetate solution in the presence of a hydrogenation catalyst.

### TECHNICAL FIELD

The present invention relates to a method for producing n-propyl acetate.

The present application claims priority on Japanese Patent Application No. 2009-277491 filed in Japan on Dec. 7, 2009, the content of which is incorporated herein by reference.

### BACKGROUND ART

Saturated esters such as n-propyl acetate, isobutyl acetate and n-butyl acetate have been commonly used as solvents and reaction solvents and are industrially important compounds. These saturated esters are typically produced by an esterification reaction resulting from condensation of a corresponding alcohol and carboxylic acid. However, in such esterification reactions, the reaction equilibrium is unable to be shifted to the product (ester) side unless the by-product in the form of water is removed outside the system, thereby it is industrially difficult to obtain a high raw material conversion rate and reaction rate. Since the latent heat of vaporization of water is much higher than that of other organic compounds, there is also the difficulty of consuming a large amount of energy when separating water by distillation.

On the other hand, unsaturated esters, which contain an unsaturated group such as an allyl group, methacrylic group or vinyl group, in the alcohol portion of an ester, can be produced industrially by going through an oxidative carboxylation reaction with a corresponding olefin and carboxylic acid.

In particular, unsaturated group-containing esters are commonly known to be able to be produced by reacting a corresponding olefin, oxygen and carboxylic acid in the presence of a palladium catalyst while in the gas phase, and there are numerous known documents regarding their production. For example, Patent Document No. 1 describes that allyl acetate can be produced industrially at an extremely high yield and high space-time yield by reacting propylene, oxygen and acetic acid in the presence of a palladium catalyst in the gas phase.

In addition, a method of adding hydrogen to the carbon-carbon double bond in unsaturated group containing esters, such as allyl acetate, that is, a hydrogenation reaction, has been disclosed in various well-known documents. For example, Patent Document No. 2 discloses a method for producing n-propyl acetate by hydrogenation of allyl acetate using a nickel catalyst as a hydrogenation catalyst. In addition, Patent Document No. 3 discloses a method for producing n-propyl acetate using a silica-supported palladium catalyst, an alumina-supported palladium catalyst, a sponge nickel, and so on.

In the Patent Document No. 3, a raw material gas containing propylene, oxygen, and acetic acid in gas phase is supplied into a reactor which is filled with palladium catalyst; outlet gas of the reactor is cooled to separate into a non-condensed component and a condensed component; a crude allyl acetate liquid which is the condensed component is distilled in a distillation tower, and thereby allyl acetate containing solution is obtained from the top of the distillation tower. After that, when the obtained allyl acetate containing solution is used as a raw material solution and hydrogenated using a hydrogenation catalyst, the target product, that is, n-propyl acetate is obtained. According to the Patent Document No. 3, an allyl acetate conversion of nearly 100% can be achieved, and the n-propyl acetate selectivity of 99.0% or more is achieved.

### PRIOR ART DOCUMENT

### Patent Document

[Patent Document No. 1] Japanese Unexamined Patent Application, First Publication No. H2-91045
[Patent Document No. 2] Japanese Unexamined Patent Application, First Publication No. H9-194427
[Patent Document No. 3] PCT International Publication No. WO 00/064852 brochure

### SUMMARY OF INVENTION

### Technical Problem to be Solved

However, according to the method disclosed in the Patent Document No. 3, that is, the method in which n-propyl acetate is produced by the hydrogenation reaction in the presence of a hydrogenation catalyst using the allyl acetate containing solution, which is obtained by propylene, oxygen, and acetic acid, as a raw material solution, the conversion of the substrate (allyl acetate) may decrease with time, and the product quality may deteriorate.

In consideration of the above-described problems, an object of the present invention is to provide a method for producing high quality n-propyl acetate by the hydrogenation reaction with a hydrogenation catalyst, using an allyl acetate containing solution as a raw material, wherein the method can prevent the gradually decrease in conversion of the substrate (allyl acetate) and the deterioration of the product quality.

### Means for Solving Technical Problem

The present inventors found that a method including a first hydrogenation step under high pressure and a second hydrogenation step for liquid phase hydrogenation using dissolved hydrogen can maintain the product quality even when the conversion of the substrate is decreased by deterioration of the hydrogenation catalyst in the first hydrogenation step. Thereby, the present inventors achieved the present invention.

That is, the present invention relates to the following inventions [1] to [6].

[1] A method for producing n-propyl acetate including:

a first hydrogenation step in which a raw material solution containing allyl acetate and a hydrogen containing gas are reacted under a pressure P₁ of 1.0 MPa G (gage pressure) or more in the presence of a hydrogenation catalyst, to hydrogenate the allyl acetate to produce a hydrogenation reaction product containing n-propyl acetate:

a gas-liquid separation step in which the hydrogenation reaction product is gas-liquid separated to produce a crude n-propyl acetate solution containing n-propyl acetate: and

a second hydrogenation step in which non-reacted allyl acetate contained in the crude n-propyl acetate solution is hydrogenated using hydrogen dissolved in the crude n-propyl acetate solution in the presence of a hydrogenation catalyst.

[2] The method for producing n-propyl acetate according to [1], wherein a hydrogenation reaction in the second hydrogenation step is a liquid phase reaction.
[3] The method for producing n-propyl acetate according to [1] or [2], wherein the pressure P₁ is in a range of 2.0 MPa G (gage pressure) to 20 MPa G (gage pressure).
[4] The method for producing n-propyl acetate according to any one of [1] to [3], wherein the ratio (P₂/P₁) between pressure P₂ in the second hydrogenation step and the P₁ is in a range of 0.9 to 2.0.
[5] The method for producing n-propyl acetate according to any one of [1] to [4], wherein the hydrogenation catalyst contains at least one metal selected from the group consisting of palladium, rhodium, ruthenium, nickel, and platinum.
[6] The method for producing n-propyl acetate according to any one of [1] to [5], wherein a reaction in the first hydrogenation step is a trickle bed type reaction.
[7] The method for producing n-propyl acetate according to any one of [1] to [6], wherein a molar ratio (Mₐ/M_{b}) between an amount (Mₐ mole) of supplied hydrogen and an amount (M_{b} mole) of supplied allyl acetate in the first hydrogenation step is in a range of 1.1 to 3.0.

### Advantageous Effects of Invention

The method for producing n-propyl acetate of the present invention is a method in which a hydrogenation reaction is performed with a hydrogenation catalyst using a raw material solution containing allyl acetate liquid. According to the production method of the present invention, it is possible to produce n-propyl acetate having a high quality, and prevent the quality of the product from deteriorating over time, which is caused by decrease in the conversion of the substrate (allyl acetate).

### BRIEF DESCRIPTION OF DRAWINGS

FIG. 1 is a diagram showing one example of a production apparatus used in the method for producing n-propyl acetate of the present invention.

FIG. 2 is a diagram showing a first hydrogenation reactor used in Example.

FIG. 3 is a diagram showing a second hydrogenation reactor used in Example.

FIG. 4 is a diagram showing a distillation purification apparatus used in Example.

### DESCRIPTION OF EMBODIMENTS

The method for producing n-propyl acetate according to the present invention is a method for producing n-propyl acetate by hydrogenation of allyl acetate, and includes at least the following steps:

a first hydrogenation step: a raw material solution containing allyl acetate and a hydrogen containing gas are reacted under a pressure P₁ of 1.0 MPa G or more in the presence of a hydrogenation catalyst, and thereby allyl acetate is hydrogenated to produce a hydrogenation reaction product containing n-propyl acetate:

a gas-liquid separation step: the hydrogenation reaction product is separated in gas and liquid to produce a crude n-propyl acetate solution: and

a second hydrogenation step: non-reacted allyl acetate contained in the crude n-propyl acetate solution is hydrogenated in the presence of a hydrogenation catalyst.

### [First Hydrogenation Step]

In the first hydrogenation step, a raw material solution containing allyl acetate and a hydrogen containing gas are reacted, and thereby allyl acetate is hydrogenated to produce a hydrogenation reaction product containing n-propyl acetate. The hydrogenation reaction is shown in the following reaction formula (1).

[Formula 1]

CH₂═CH—CH₂—OCOCH₃+H₂→CH₃—CH₂—CH₂—OCOCH₃ (1)

The mode of the hydrogenation reaction in the first hydrogenation step in the present invention is a gas-liquid reaction between the raw material solution containing allyl acetate and a hydrogen containing gas.

The mode of the hydrogenation reaction can be roughly divided into three categories, that is, gas phase reactions, liquid phase reactions, and gas-liquid reactions. Among these, the gas phase reaction has a problem that a large amount of energy is necessary to evaporate a substrate or a solvent. In the liquid phase reaction, the substrate is hydrogenated by hydrogen (H₂) dissolved in a reaction solvent containing the substrate. The liquid phase reaction is effective when the solubility of hydrogen is high. However, when sufficient solubility of hydrogen to the concentration of the substrate is not obtained, it is necessary to increase the solubility of hydrogen gas by increasing the pressure of hydrogen gas, or rising the temperature (The higher the temperature is, the higher the solubility of hydrogen gas is. This is adversely phenomenon to those of normal gases). In the gas-liquid reaction, the substrate and the solvent as a liquid phase and hydrogen as a gas phase are reacted. The gas-liquid reaction does not need a large amount of energy, such as vaporization heat, and does not relate to the solubility of hydrogen to the substrate or the solvent. Therefore, the gas-liquid reaction is extremely useful reaction mode.

It is preferable that the mode of the hydrogenation reaction in the first hydrogenation step be a trickle bed type reaction in which gas (the hydrogen containing gas) is a continuous phase, and liquid (the raw material solution) is a discontinuous phase, on a solid catalyst (hydrogenation catalyst).

It is preferable that the raw material solution be obtained by diluting allyl acetate with an inert solvent. Due to the reaction heat is high, when allyl acetate having a high purity is used in the hydrogenation reaction without dilution and the conversion of the substrate is 100%, the temperature rises considerably in the reactor. Therefore, it is virtually impossible to use allyl acetate industrially without dilution.

As the inert solvent, an organic solvent which does not contain an ethylenical carbon-carbon double bond is preferable, because this is rarely affected with the hydrogenation reaction.

Examples of the organic solvent which does not contain an ethylenical carbon-carbon double bond include saturated esters such as ethyl acetate, n-propyl acetate, butyl acetate, isopropyl acetate, n-propyl propionate, ethyl propionate, butyl propionate, and isopropyl propionate; hydrocarbons such as cyclohexane, n-hexane, and n-heptane; aromatic hydrocarbons, such as benzene, and toluene; ketones such as acetone, and methyl ethyl ketones; halogenated hydrocarbons such as carbon tetrachloride, chloroform, methylene chloride, and methyl chloride; ethers such as diethyl ether, and di-n-propyl ether; alcohols such as ethanol, n-propanol, isopropanol, n-butanol, and sec-butanol; and amides such as N-methyl-2-pyrrolidone, and N,N-dimethyl acetamide.

Among these, saturated esters, hydrocarbons, and ketones are preferable, because they are rarely hydrogenated, and rarely cause the hydrogenolysis reaction of allyl acetate.

N-propyl acetate produced by the hydrogenation reaction is inactive in the hydrogenation reaction. Therefore, it is preferable that a part of n-propyl acetate obtained by the hydrogenation reaction in the first hydrogenation step is cyclically used as the dilution solvent.

The concentration of allyl acetate in the raw material solution is preferably in a range of 1% by mass to 50% by mass, more preferably in a range of 3% by mass to 30% by mass, and most preferably in a range of 5% by mass to 15% by mass.

When the concentration of allyl acetate in the raw material solution is less than 1% by mass, remarkable temperature rise due to exothermal reaction can be sufficiently prevented, but the productivity may decrease because the concentration of allyl acetate is too low. In contrast, when is exceeds 50% by mass, it is difficult to prevent a remarkable temperature rise due to heat production in the hydrogenation reaction. In addition, when an adiabatic reactor is used as the hydrogenation reactor in the first hydrogenation step, there is high possibility that the temperature of the reactor cannot be controlled. Specifically, it may be impossible to control the temperature of the reactor to be in a range of 0° C. to 200° C.

Examples of a method for producing allyl acetate include (1) a method in which a raw material gas containing propylene, oxygen, and acetic acid are reacted in gas phase in the presence of a palladium catalyst, (2) a method in which propylene chloride, and carboxylic acid or the salt thereof are reacted, and (3) a method in which allyl alcohol is esterified with carboxylic acid by condensation. Among these methods, the method (1) is preferable because allyl acetate can be produced using a simple apparatus with low cost and high efficiency.

Any hydrogen containing gas can be used without any limitation as long as containing hydrogen gas. Any available commercial gas can be also used. Hydrogen gas having a high purity is preferably used. Hydrogen obtained as naphtha cracker fractions can also be used. In this case, the hydrogen containing gas may contain impurities such as methane.

It is preferable that the amount of the hydrogen containing gas supplied in the first hydrogenation step be adjusted such that a molar ratio (Mₐ/M_{b}) between an amount (Mₐ mole) of hydrogen supplied and an amount (M_{b} mole) of allyl acetate supplied be in a range of 1.1 to 3.0, more preferably in a range of 1.1 to 2.0. In theory, the molar ratio (Mₐ/M_{b}) is sufficiently 1.0 or more. However, when it is 1.0 or less, and a side reaction, such as a hydrogenolysis reaction is caused, the amount of hydrogen for the hydrogenation reaction may be insufficient due to the side reaction. In contrast, when it exceeds 3.0, there is too much hydrogen containing gas which is an economical disadvantage.

As the hydrogen catalyst, hydrogen catalysts include groups 8, 9 and 10 elements of the periodic table (IUPAC Inorganic Chemistry Nomenclature Revised Edition, 1989, to apply similarly hereinafter) are preferable. Specifically, the preferable hydrogen catalyst contains at least one metal selected from the group consisting of iron, ruthenium, osmium, cobalt, rhodium, iridium, nickel, palladium and platinum. In addition, the hydrogen catalyst containing at least one metal selected from the group consisting of palladium, rhodium, ruthenium, nickel, and platinum is more preferable. Furthermore, the hydrogen catalyst containing at least one metal selected from the group consisting of palladium, rhodium, and ruthenium is most preferable.

When a fixed-bed reactor is used in the hydrogenation reaction of the first hydrogenation step, the hydrogen catalyst is preferably a catalyst in which the metal is supported on a support, because the catalyst can make a contact area between the hydrogenation catalyst and allyl acetate large, and improve the contact efficiency. Moreover, the hydrogenation catalyst may be the metal, or compounds thereof,

A substance normally used as a catalyst support (such as a porous substance) can be used without limitation for the hydrogenation catalyst support. Examples of such a support include silica, alumina, titanium oxide, diatomaceous earth, carbon and mixtures thereof.

Although there are no particular limitations on the specific surface area of the support, from the viewpoint of facilitating high dispersion of catalyst metal, it is preferable to use a support having a large specific surface area. Specifically, the value of specific surface area as determined according to the BET method is preferably in a range of 10 m²/g to 1,000 m²/g, more preferably in a range of 30 m²/g to 800 m²/g, and most preferably in a range of 50 m²/g to 500 m²/g.

In addition, although there are also no particular limitations on the total pore volume of the support, it is preferably in a range of 0.05 ml/g to 6.5 ml/g, more preferably in a range of 0.1 ml/g to 5.0 ml/g and particularly preferably in a range of 0.5 ml/g to 3.0 ml/g.

There are no particular limitations on the form of the support, and the shape can be suitably selected from commonly known forms. From the viewpoint of uniformity of the internal pressure of the hydrogenation reactor, pellets, spheres, hollow cylinders, wheels with spokes, a honeycomb type monolithic support having parallel flow channels or foam ceramic support with high porosity are preferable, and pellets or spheres are particularly preferable in consideration of ease of production.

It is preferable that the support can inhibit remarkable decrease of pressure when bulk loading onto a catalyst layer, and have an extremely large geometrical surface area as compared with total bulk volume. In consideration of these points, the support preferably has an external size in a range of 0.5 mm to 5.0 mm and more preferably in a range of 1.0 mm to 4.5 mm.

The reaction temperature during the hydrogenation reaction in the first hydrogenation step varies depending on the concentration of allyl acetate in the raw material solution, etc. However, the reaction temperature is preferably in a range of 0° C. to 200° C., and more preferably in a range of 40° C. to 150° C. When the reaction temperature is less than 0° C., it is difficult to obtain a sufficient reaction rate. In contrast, when the reaction temperature exceeds 200° C., the hydrogenolysis reaction, which is the side reaction, is easily caused.

The pressure P₁ of the hydrogenation reaction in the first hydrogenation step is 1.0 MPa G or more, preferably 1.5 MPa G or more, and more preferably 2.0 MPa G or more. Moreover, “G” means “gage pressure” in the present description.

When the pressure P₁ is 1.0 MPa G or more, the hydrogenation reaction is promoted, the side reactions for producing acetic acid due to hydrogenolysis can be inhibited, and the selectivity of n-propyl acetate can be improved. In addition, the concentration of dissolved hydrogen in the reaction solution increases. Thereby, it is possible to retain a sufficient amount of hydrogen for hydrogenation of non-reacted allyl acetate, and 1-propenyl acetate (cis and trans) in the second hydrogenation step. The upper limit of the pressure P₁ is not particularly limited. However, the upper limit of P₁ G is preferably 20 MPaG, from the viewpoint of pressure resistance, the cost of the reactor, and safety.

### [Gas-Liquid Separation Step]

The reaction product containing n-propyl acetate obtained in the first hydrogenation step contains condensed components such as the produced n-propyl acetate, the non-reacted allyl acetate, 1-propenyl acetate (cis and trans) produced by isomerizing allyl acetate, acetic acid which is produced by the side reaction (hydrogenolysis), and non-condensed components such as non-reacted hydrogen, C3 gas (hydrocarbon gas having three carbon atoms) produced by the side reaction (hydrogenolysis) and methane which is commonly contained in hydrogen used industrially. In the gas-liquid separation step, the reaction product is cooled to separate into the condensed component and the non-condensed component.

The separated non-condensed component may be removed by burning or reused in the hydrogenation reaction by increasing the pressure with a compressor. Meanwhile, it is preferable that a part of the condensed component be reused as a diluent solvent to the raw material solution, and the residue be supplied into the second hydrogenation step.

Moreover, the condensation of the reaction product in the gas-liquid separation step may be carried out using a well-known gas-liquid separation apparatus.

The temperature in the gas-liquid separation step is preferably in a range of 20° C. to 80° C.

The pressure in the gas-liquid separation step is preferably equal to the pressure P₂ in the second hydrogenation step, and more preferably in a range of 0.9 MPa G to 20 MPa G, from an economic standpoint.

### [Second Hydrogenation Step]

In the second hydrogenation step, the non-reacted allyl acetate and 1-propenyl acetate (cis and trans), which are contained in the condensed component containing n-propyl acetate and separated in the gas-liquid separation step, are hydrogenated. The reaction mode in the hydrogenation reaction in the second hydrogenation step is not limited. The reaction mode may be a gas reaction, or a gas-liquid reaction. However, the reaction mode of the hydrogenation reaction in the second hydrogenation step is basically a liquid reaction in which hydrogenation is carried out using dissolved hydrogen in the crude n-propyl acetate solution in the presence of a hydrogenation catalyst.

When the reaction mode of the hydrogenation reaction is a gas-liquid reaction, hydrogen which is not dissolved in the liquid phase may be in the gas phase. The concentration of dissolved hydrogen in the crude n-propyl acetate solution is determined by the operation temperature, the operation pressure, the concentration of hydrogen in the gas phase, the composition of the liquid phase in the gas-liquid separation step.

The total concentration of allyl acetate and 1-propenyl acetate in the crude n-propyl acetate solution in the second hydrogenation step varies depending on the reaction efficiency in the first hydrogenation step. However, the total concentration is preferably 3% by mass or less, more preferably 2% by mass or less, and most preferably 1% by mass or less. In order to completely hydrogenate allyl acetate, and 1-propenyl acetate, in speculation, an amount in term of mole of hydrogen is necessary, which is equal to or more an amount in terms of mole of allyl acetate, and 1-propenyl acetate. However, when the total concentration of allyl acetate, and 1-propenyl acetate in the crude n-propyl acetate solution exceeds 3% by mass, and the hydrogen solubility which is necessary to supply hydrogen into the hydrogenation reaction is achieved, a high pressure in the reaction system is required.

The solubility (Journal of chemical engineering data, vol. 32, No. 1, 1987, P.23) of hydrogen gas into n-propyl acetate at 18° C. is shown in Table 1. For example, solubility of hydrogen at 4.09 MPa of a reaction system pressure is 1.66% by mole. That is, in order to improve hydrogen solubility, remarkably high pressure is required. The higher the total concentration of allyl acetate and 1-propenyl acetate is, the more difficult the hydrogenation by liquid phase hydrogenation is.

TABLE 1

Pressure [MPa]

| | 1.59 | 2.35 | 3.1 | 4.09 |
|---|---|---|---|---|
| H₂ solubility [mol %] | 0.73 | 0.98 | 1.29 | 1.66 |

The reaction temperature in the second hydrogenation step is preferably in a range of 0° C. to 200° C., and more preferably in a range of 40° C. to 150° C. When the reaction temperature is less than 0° C., it is difficult to obtain a sufficient reaction rate. In contrast, when the reaction temperature exceeds 200° C., hydrogenolysis easily occurs.

The pressure P₂ in the second hydrogenation step is not limited as long as obtaining sufficient hydrogen solubility for the hydrogenation reaction of allyl acetate and 1-propenyl acetate in the crude n-propyl acetate solution. Moerover, the pressure ratio (P₂/P₁) between the pressure P₂ in the second hydrogenation step and the pressure P₁ in the first hydrogenation step is preferably in a range of 0.9 to 2.0. When the pressure ratio (P₂/P₁) is 0.9 or more, sufficient hydrogenation solubility in the hydrogenation reaction is easily obtained. Thereby, allyl acetate and 1-propenyl acetate in the crude n-propyl acetate solution are easily and sufficiently hydrogenated. When the pressure ratio (P₂/P₁) is 2.0 or less, the facility cost can be reduced, and the economic efficiency is improved. Specifically, when pressure resistance of the reactor, and cost are concerned, the pressure P₂ in the second hydrogenation step is preferably in a range of 0.9 MPa G to 20 MPa G, more preferably in a range of 1.3 MPa G to 20 MPa G, and most preferably in a range of 1.8 MPa G to 20 MPa G.

In the present invention, from the viewpoint of economical efficiency, it is preferable that the hydrogenation reaction in the second hydrogenation step was carried out, while the pressure in the gas-liquid separation step was adjusted to P₂ and the pressure in the second hydrogenation step was maintained to P₂. However, after the gas-liquid separation step, the second hydrogenation step may be carried out by increasing the pressure to P₂ using a pump.

In the second hydrogenation step, dissolved hydrogen in the crude n-propyl acetate solution obtained in the gas-liquid separation step is used to the hydrogenation reaction. However, it is also possible to add hydrogen from the outside of the reaction system. Any method for additionally introducing hydrogen can be used without limitations. Examples of the method for additionally introducing hydrogen include a method for adding hydrogen into the condensed component (crude n-propyl acetate solution) in the gas-liquid separation step, a method for adding hydrogen in the second hydrogenation step, a method for introducing hydrogen through a liquid supplying pipe between the gas-liquid separation step and the second hydrogenation step, and a method for providing hydrogen into a dissolution tank and introducing hydrogen into the dissolution tank.

As the hydrogenation catalyst in the second hydrogenation step, the hydrogenation catalyst listed in the first hydrogenation step can be used, and preferable embodiment of the catalysts are the same as those in the first hydrogenation step. As the hydrogenation catalyst in the second hydrogenation step, a same catalyst used in the first hydrogenation step can be used, and also a different catalyst can be used, too.

The hydrogenation reaction solution obtained in the second hydrogenation step contains a small amount of high boiling point components such as acetic acid, and propyl propionate, and low boiling point components such as C3 gas, propionaldehyde, and moisture. Therefore, the hydrogenation reaction solution obtained in the second hydrogenation step is preferably purified by distillation. The distillation is carried out using a well-known distillation tower. For example, a distillation bottom solution containing a large amount of the high boiling point components, such as acetic acid and propyl propionate are extracted from the bottom of the distillation tower. A distillation top solution containing a large amount of the low boiling point components, such as C3 gas, propionaldehyde, and moisture is extracted from the top of the distillation tower. N-propyl acetate (product) having a high purity are extracted from the intermediate of the distillation tower. Thereby, n-propyl acetate product having a high purity can be produced.

The total concentration of allyl acetate and 1-propenyl acetate contained in the n-propyl acetate after purification is preferably 1,000 ppm by mass or less, more preferably 500 ppm by mass or less, and most preferably 100 ppm by mass or less.

[Production Apparatus for n-Propyl Acetate]

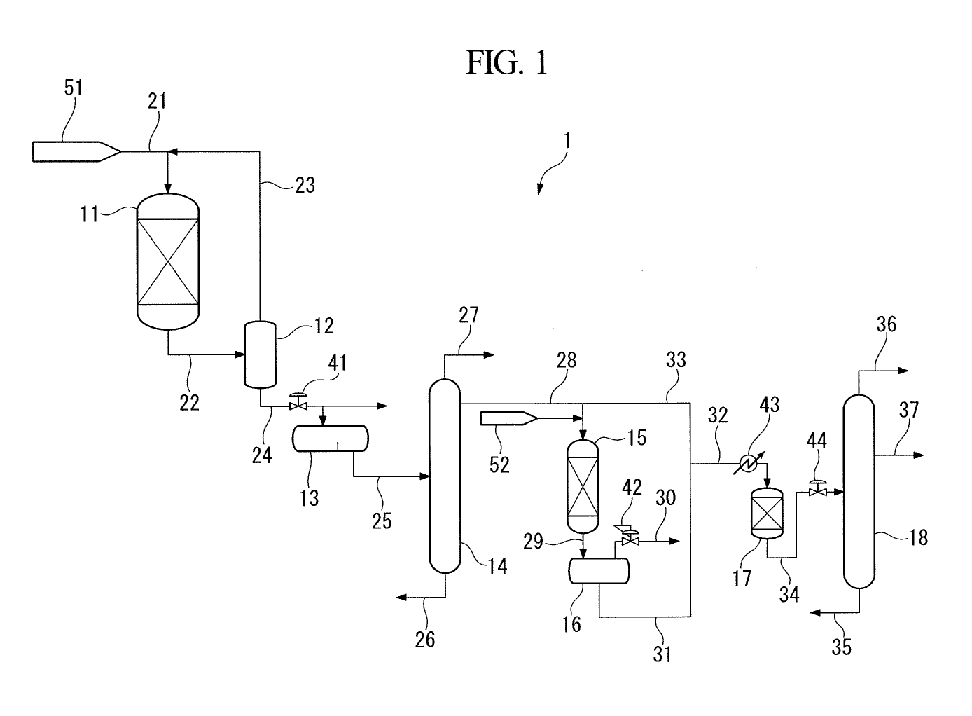

Below, one embodiment of the method for producing n-propyl acetate according to the present invention is explained referring to FIG. 1. FIG. 1 is a diagram showing one example of a production apparatus used in the method for producing n-propyl acetate of the present invention.

As shown in FIG. 1, the production apparatus 1 includes the reactor 11 in which a raw material gas 51 containing propylene, oxygen, and acetic acid is reacted to produce allyl acetate; a condensed component separation tank 12 in which the outlet gas of the reactor 11 is condensed to obtain the condensed component (crude allyl acetate solution) containing allyl acetate; an oil-water separation tank 13 in which an oil component containing a large amount of allyl acetate is separated from the crude allyl acetate solution supplied from the condensed component separation tank 12; a first distillation tower 14 in which the oil component containing a large amount of allyl acetate supplied from the oil-water separation tank 13 is distilled; a first hydrogenation reactor 15 in which the hydrogen containing gas 52 is reacted with the raw material solution which is a diluted allyl acetate solution having a high purity supplied from the fist distillation tower 14 with an inert solvent in the presence of the hydrogenation catalyst in order to hydrogenate allyl acetate; a gas-liquid separation tank 16 in which the reaction product supplied from the first hydrogenation reactor is separated into the condensed component (crude n-propyl acetate solution) containing n-propyl acetate and the non-condensed component; a second hydrogenation reactor 17 in which non-reacted allyl acetate and 1-propenyl acetate contained in the crude n-propyl acetate solution are hydrogenated in the presence of the hydrogenation catalyst; and a second distillation tower 18 in which the hydrogenation reaction solution supplied from the second hydrogenation reactor 17 is distilled.

In addition, the production apparatus 1 further includes a flow path 21 for supplying the raw material gas 51 into the reactor 11; a flow path 22 for extracting an outlet gas of the reactor 11 and supplying the gas into the condensed component separation tank 12; a flow path 23 for returning the non-condensed component from the condensed component separation tank 12 into the reactor 11; a flow path 24 having a control valve 41 for supplying the crude allyl acetate solution from the condensed component separation tank 12 into an oil-water separation tank 13; a flow path 25 for supplying an oil component containing a large amount of allyl acetate from the oil-water separation tank 13 into the first distillation tower 14; a flow path 26 for extracting the distillation tower bottom solution from the first distillation tower 14; a flow path 27 for extracting the distillation tower top solution from the first distillation tower 14; a flow path 28 for supplying an allyl acetate solution having a high purity from the first distillation tower 14 into the first hydrogenation reactor 15; a flow path 29 for supplying the hydrogenation reaction product from the first hydrogenation reactor 15 into the gas-liquid separation tank 16; a flow path 30 having a secondary pressure control valve 42 for extracting the non-condensed component from the gas-liquid separation tank 16; a flow path 31 for extracting the crude n-propyl acetate solution which is the condensed component, from the gas-liquid separation tank 16; a flow path 32 which is branched from the flow path 31, has a heat exchanger 43, for supplying the crude n-propyl acetate solution from the gas-liquid separation tank 16 into the second hydrogenation reactor 17; a flow path 33 which is branched from the flow path 31 and joined to the flow path 28, for reusing a part of the crude n-propyl acetate solution extracted from the gas-liquid separation tank 16 as the inert solvent; a flow path 34 having a control valve 44 for supplying the hydrogenation reaction solution from the second hydrogenation reactor 17 into the second distillation tower 18; a flow path 35 for extracting the distillation tower bottom solution from the second distillation tower 18; a flow path 36 for extracting the distillation top solution from the second distillation tower 18; and a flow path 37 for extracting an n-propyl acetate product from the second distillation tower 18.

### [Production Step for Allyl Acetate]

The raw material gas 51 containing propylene, oxygen and acetic acid, which is supplied from the flow path 21, and the recycle gas from the flow path 23 are combined and supplied into the reactor 11 which is filled with the catalyst, and then allyl acetate is produced in accordance with the reaction represented by the following formula (2).

[Formula 2]

CH₂═CH—CH₃+½O₂+CH₃COOH→CH₂═CH—CH₂—OCOCH₃+H₂O (2)

There are no particular limitations on the propylene contained in the raw material gas 51. Although the propylene containing lower saturated hydrocarbons such as propane or ethane may also be acceptable, the highly pure propylene is preferably used.

Moreover, there are also no particular limitations on the oxygen in the raw material gas 51. The oxygen may be diluted with an inert gas such as nitrogen or carbon dioxide gas, and air, for example, may be used. However, as shown in FIG. 1, in the case of circulating the non-reacted gas as the non-condensed component through the flow path 23, highly pure oxygen, and particularly oxygen having a purity of 99% or more, is preferably used.

The content of acetic acid in the raw material gas 51 is preferably in a range of 4% by volume to 20% by volume, and more preferably in a range of 6% by volume to 10% by volume.

The content of propylene in the raw material gas 51 is preferably in a range of 5% by volume to 50% by volume, and more preferably in a range of 10% by volume to 40% by volume.

The molar ratio (M_{c}:M_{d}:Mₑ) between acetic acid (amount of material: M_{c}), propylene (amount of material: M_{d}), and oxygen (amount of material: Mₑ) is preferably in a range of 1:0.25 to 13:0.15 to 4, and more preferably in a range of 1:1 to 7:0.5 to 2.

The raw material gas 51 supplied into the reactor 11 may contain a diluent such as nitrogen, carbon dioxide, and rare gas, in addition to acetic acid, propylene, and oxygen, if necessary.

Any catalyst may be used for the catalyst filled in the reactor 11 as long as it has the ability to obtain allyl acetate by reacting propylene, acetic acid and oxygen described by the formula (2). The catalyst is preferably a supported solid catalyst containing the following components (α) to (γ):

(α) palladium;
(β) a compound having at least one element selected from the group consisting of copper, lead, ruthenium and rhenium; and
(γ) at least one compound selected from the group consisting of an alkaline metal acetate and an alkaline earth metal acetate.

Although any valence of palladium may be used for the component (α), metal palladium is preferable. The “metal palladium” as referred to here is palladium having a valence of 0. Metal palladium can be typically obtained by reducing palladium ions having a valence of 2 and/or 4 using a reducing agent in the form of hydrazine or hydrogen and the like. At this time, it is not necessary for the component (α) that all of the palladium be in the metal state.

The component (α) may not be limited to metal palladium. A palladium salt able to be converted to metal palladium can also be used. Examples of palladium salts which can be converted to metal palladium include, palladium chloride, palladium sodium chloride, palladium nitrate, palladium sulfate and the like.

The mass ratio (W_{δ}:W_{α}) between the support (mass: W_{δ}) and the component (α)(mass: W_{α}) is preferably in a range of 1:0.1 to 5.0, and more preferably in a range of 1:0.3 to 1.0.

A water soluble salt such as a nitrate, carbonate, sulfate, organic acid salt or halide having at least one element selected from the group consisting of copper, lead, ruthenium and rhenium can be used for the component (β). Among these, chlorides are preferable since they are easy to obtain and have superior water solubility. In addition, a preferable example of an element among the aforementioned elements is “copper”. Examples of copper salts include cuprous chloride, cupric chloride, copper acetate, copper nitrate, copper acetylacetonate, copper sulfate and the like.

The mass ratio (W_{α}:W_{β}) between the component (α) (mass: W_{α}) and the component (β) (mass: W_{β}) is preferably in a range of 1:0.05 to 10, and more preferably in a range of 1:0.1 to 5.

A preferable example of the component (γ) is an alkaline metal acetate, specific examples of which include lithium acetate, sodium acetate, and potassium acetate and the like. Among these sodium acetate and potassium acetate are more preferable, while the potassium acetate is the most preferable.

Although there are no particular limitations on the loading amount of the component (γ), the loaded amount is preferably in a range of 1% by mass to 30% by mass based on 100 parts by mass of the support.

In addition, in order to achieve a desired loading amount, an alkaline metal acetate may be added to reactor 11 by a method such as adding it to the raw material gas in the form of an aqueous solution or an acetic acid solution.

There are no particular limitations on the support used to support the catalyst component, and may be a porous substance typically used as a support. Preferable examples of supports include silica, alumina, silica-alumina, diatomaceous earth, montmorillonite and titania, while silica is more preferable. In addition, there are no particular limitations on the form of the support. Specific examples of support forms include powders, spheres, pellets and the like.

Although there are no particular limitations on the particle diameter of the support, it is preferably in a range of 1 mm to 10 mm and more preferably in a range of 3 mm to 8 mm. In the case that the particle diameter of the support is 1 mm or more, and when the tubular reactor is used as the reactor 11, and the raw material gas circulates in the reactor filled with the catalyst, it is possible to circulate effectively the raw material gas without causing large pressure loss. When the particle diameter is 10 mm or less, the raw material gas can be diffused inside the catalyst, and thereby the catalyst reaction is easily carried out.

The pore structure of the support is such that the pore diameter is preferably in a range of 1 nm to 1,000 nm and more preferably in a range of 2 nm to 800 nm.

There are no particular limitations on the method used to support the components (α) to (γ) onto the support, and any method may be used.

More specifically, a method may be used in which the support is impregnated into an aqueous solution containing the component (α) in the form such as a palladium salt and the component (β), and then the support is treated with an aqueous solution of an alkaline metal salt. At this time, alkaline treatment is preferably carried out without drying the support in which the catalyst liquid is impregnated. The treating time with an aqueous solution of an alkaline metal salt is the amount of time required for the salt of the catalyst component impregnated in the support to be completely converted to a compound insoluble in water, and normally 20 hours is adequate.

Next, the metal salt of the catalyst component precipitated on the surface layer of the catalyst support by the alkali treatment is treated with a reducing agent to obtain a metal of valence zero. The reduction is carried out in a liquid phase by adding a reducing agent such as hydrazine or formalin. Subsequently, the catalyst support is rinsed with water until chlorine ions and the like are no longer detected, followed by drying, supporting an alkaline metal acetate and drying further.

There are no particular limitations on the reaction type between propylene, oxygen, and acetic acid in the reactor 11, and a known reaction type of the prior art can be selected. In general, it is preferable to select an optimal reaction type depending on the kind of the catalyst used. For example, in the case of using a supported solid catalyst, a fixed bed flow reaction in which the catalyst is filled into the reactor 11 is preferable.

Although there are no particular limitations on the material of the reactor 11, the reactor 11 preferably includes a material having corrosion resistance.

There are no particular limitations on the reaction temperature in the reactor 11, and the temperature is preferably in a range of 100° C. to 300° C. and more preferably in a range of 120° C. to 250° C.

Although there are no particular limitations on the reaction pressure, from the viewpoint of simplicity of the equipment, a pressure is preferably in the range of 0.0 MPa G to 3.0 MPa G and more preferably in the range of 0.1 MPa G to 1.5 MPa G.

The space velocity at the normal condition of the raw material gas passing through the catalyst is preferably in a range of 10 hour⁻¹ to 15,000 hour⁻¹, and more preferably in a range of 300 hour⁻¹ to 8,000 hour⁻¹.

The gas containing allyl acetate produced in the reactor 11 is extracted from the outlet of the reactor 11 into the flow path 22 as the outlet gas, and supplied into the condensed component separation tank 12. In the condensed component separation tank 12, the outlet gas is condensed and the crude allyl acetate solution containing mainly condensed components such as allyl acetate, acetic acid, and water is extracted into the flow path 24 from the bottom of the condensed component separation tank 12. In addition, it is preferable that the non-condensed component containing mainly propylene, oxygen, and carbon dioxide be extracted from the top of the condensed component separation tank 12 and returned into the reactor 11 via the flow path 23.

In the present invention, an absorption tower for changing acetic acid and water to an absorbing liquid may be provided instead of the condensed component separation tank 12.

The crude allyl acetate solution extracted into the flow path 24 is purified in the oil-water separation tank 13 and the first distillation tower 14 to remove impurities such as side reaction products.

Specifically, a part or all of the crude allyl acetate solution extracted into the flow path 24 is supplied into the oil-water separation tank 13, and the oil layer containing a large amount of allyl acetate is separated, extracted into the flow path 25, and supplied into the first distillation tower 14. By distillation in the first distillation tower 14, the distillation tower bottom solution containing a large amount of high boiling point components such as acetic acid, acrylic acid, allyl acrylate, and diacetate is extracted into the flow path 26. The distillation tower top solution containing a large amount of low boiling point components such as acrolein, propionaldehyde, and water is extracted into the flow path 27. Thereby, the high boiling point components and the low boiling point components are removed. The tower bottom solution and tower top solution extracted and removed into the flow paths 26 and 27 may be used as a boiler fuel, or reused to produce allyl acetate.

The distillation tower intermediate solution containing mainly allyl acetate, that is, an allyl acetate solution having a high purity, is produced from the intermediate stage of the first distillation tower 14.

The purity of allyl acetate in the allyl acetate solution having a high purity is commonly 95% or more. The total concentration of acrolein, propionaldehyde, 2-methylcrotone aldehyde, acrylic acid, and allyl acrylate which are contained in the allyl acetate solution having a high purity varies depending on the reaction conditions in the allyl acetate production step, and the distillation conditions in the first distillation tower 14. However, the total concentration is preferably in a range of 0% by mass to 5% by mass, more preferably in a range of 0% by mass to 3% by mass, and most preferably in a range of 0% by mass to 1% by mass.

### [First Hydrogenation Step]

The allyl acetate solution having a high purity which is extracted from the first distillation tower 14 into the flow path 28, is mixed with the diluent solvent from the flow path 33 to be diluted, and then it is supplied into the first hydrogenation reactor 15 which is filled with the hydrogenation catalyst as the raw material solution of the hydrogenation reaction.

It is preferable that the first hydrogenation reactor 15 be a fixed-bed reactor having a catalyst bed filled with the hydrogenation catalyst. The first hydrogenation reactor 15 may be an adiabatic reactor.

In the first hydrogenation reactor 15, allyl acetate contained in the raw material solution is hydrogenated with hydrogen contained in the hydrogen containing gas 52 at the pressure P₁ in the presence of the hydrogenation catalyst, and produces n-propyl acetate.

In this embodiment, the hydrogenation reaction is carried out in the first hydrogenation reactor 15. The condensed component (crude n-propyl solution) containing n-propyl acetate separated in the gas-liquid separation tank 16, which is explained below, is extracted from the flow path 31. Then, a part of the extracted condensed component is combined with the allyl acetate solution having a high purity in the flow path 33 and passes through the flow path 28, and then used as the diluent solvent in the hydrogenation reactor 15. Due to the reaction heat of allyl acetate is very large, when allyl acetate having a high purity produced in the first distillation tower 14 is used in the hydrogenation reaction without dilution, and the conversion of the substrate is 100%, the temperature rises considerably in the first hydrogenation reactor 15. Therefore, it is impossible industrially to use allyl acetate without dilution in practical. However, it is possible to mix the allyl acetate solution having a high purity with the inert solvent, which is separately prepared, and used the obtained mixture as the raw material solution, without reusing the crude n-propyl acetate solution as the diluent solvent.

### [Gas-Liquid Separation Step]

The hydrogenation reaction product containing n-propyl acetate is supplied from the first hydrogenation reactor 15 into the flow path 29, and supplied into the gas-liquid separation tank 16. In the gas-liquid separation tank 16, the hydrogenation reaction product is separated into the condensed component containing the produced n-propyl acetate, non-reacted allyl acetate, 1-propenyl acetate (cis and trans) which allyl acetate is isomerized, acetic acid which is produced by the side reaction (hydrogenolysis), and the non-condensed components such as non-reacted hydrogen, C3 gas produced by the side reaction (hydrogenolysis) and methane which is commonly contained in hydrogen used industrially.

The pressure in the gas-liquid separation tank 16 is preferably the same as the pressure P₂ in the second hydrogenation reactor 17.

In addition, a flow path for further adding hydrogen may be provided with the gas-liquid separation tank 16, if necessary.

The separated non-condensed components are extracted into the flow path 30 using the secondary control valve 42, and supplied into flare in order to burn and remove. Otherwise, the non-condensed components are supplied again into the first hydrogenation reactor 15 and reused by rising the pressure again with a compressor. On the other hand, a part of the n-propyl acetate solution, which is the condensed component, is reused as the diluent solvent, and the rest is supplied into the second hydrogenation reactor 17 via the flow path 32.

A hydrogen dissolution tank for further adding hydrogen into the crude n-propyl acetate solution may be provided between the gas-liquid separation tank 16 and the second hydrogenation reactor 17, if necessary.

### [Second Hydrogenation Step]

In the second hydrogenation reactor 17, the non-reacted allyl acetate and 1-propenyl acetate (cis and trans) contained in the crude n-propyl acetate solution are hydrogenated at the pressure P₂ in the presence of the hydrogenation catalyst using dissolved hydrogen in the crude n-propyl acetate solution supplied from the gas-liquid separation tank 16. The reaction in the second hydrogenation reactor 17 is basically a liquid phase reaction. While passing the crude n-propyl acetate solution containing dissolved hydrogen gas through the catalyst bed (fixed bed), the hydrogenation reaction is carried out. However, the reaction in the second hydrogenation reactor 17 is not limited to a liquid phase reaction. The reaction may be a gas phase reaction, or a gas-liquid reaction using as a bubble tower. In the case of a gas-liquid reaction, hydrogen, which cannot be dissolved in a liquid phase, may be in a gas phase in the reactor.

It is possible to heat the crude n-propyl acetate solution supplied from the gas-liquid separation tank 16 using the heat exchanger 43, in order to promote the hydrogenation reaction in the second hydrogenation reactor 17. When the temperature of the crude n-propyl acetate solution in the gas-liquid separation tank 16 is higher than the temperature in the hydrogenation reaction in the second hydrogenation reactor 17, it is also possible to cool to an appropriate temperature of the crude n-propyl acetate solution by the heat exchanger 43.

### [Purification Step]

The hydrogenation reaction product solution obtained in the second hydrogenation reactor 17 is extracted into the flow path 34, and supplied into the second distillation tower 18. In the second distillation tower 18, the hydrogenation product solution is distillated. Thereby, the distillation tower bottom solution containing a large amount of high boiling point components such as acetic acid, and propyl propionate is extracted into the flow path 35. The distillation tower top solution containing a large amount of low boiling point components such as C3 gas, propionaldehyde, and moisture is extracted into the flow path 36, and removed. Then, n-propyl acetate (product) having a high purity is extracted into the flow path 37 at the intermediate stage in the second distillation tower 18.

As explained above, in the production apparatus 1, allyl acetate, which is produced by propylene, oxygen, and acetic acid, is hydrogenated; the allyl acetate containing solution having a high purity, which is purified, is supplied in the first hydrogenation reactor as the raw material solution to carry out the hydrogenation reaction at high pressure with high selectivity; and then, in the second hydrogenation reactor (liquid phase reactor), the non-reacted products are hydrogenated with the dissolved hydrogen; and thereby the obtained hydrogenation reaction solution is distilled to produce n-propyl acetate having a high purity.

According to the production method for n-propyl acetate according to the present invention, n-propyl acetate having a high purity can be obtained. That is, it is possible to prevent deterioration in the product quality even when the hydrogenation catalyst in the first hydrogenation step is deactivated with time, and the conversion rate of the substrate (allyl acetate) decreases.

In the hydrogenation reaction of allyl acetate using the hydrogenation catalyst, when the catalysis activity decreases, the conversion rate of allyl acetate also decreases. As a result, allyl acetate, which is the substrate, and 1-propyl acetate (cis and trans) obtained by isomerization of allyl acetate are contaminated in the reaction product. The boiling point of the contaminated allyl acetate and 1-propenyl acetate (cis and trans) is similar to that of n-propyl acetate. Therefore, the separation and purification of n-propyl acetate is difficult. This causes a decrease of purification of an n-propyl acetate product.

In contrast, according to the method for producing n-propyl acetate of the present invention, allyl acetate is hydrogenated with a high selectivity under high pressure in the first hydrogenation step, the non-reacted components are hydrogenated using dissolved hydrogen in the crude n-propyl acetate solution in the second hydrogenation step, and thereby n-propyl acetate having a high purity is produced with a decrease in the amount of allyl acetate and 1-propyl acetate. In this way, n-propyl acetate having a high purity is produced by including the second hydrogenation step after the first hydrogenation step. In addition, it is also possible to prevent the decrease of the conversion ratio of the substrate (allyl acetate) even when the hydrogenation catalyst in the first hydrogenation step is deactivated with time. Due to this, it is possible to prevent the deterioration of quality of the n-propyl acetate produced.

Moreover, the method for producing n-propyl acetate according to the present invention is not limited to the embodiment above. For example, it is possible to include a third hydrogenation step which is similar to the second hydrogenation step after the second hydrogenation step.

In addition, the apparatus used in the method for producing n-propyl acetate according to the present invention is not limited to the production apparatus 1 above. The apparatus used in the method for producing n-propyl acetate according to the present invention may only include the apparatus subsequent to the first hydrogenation reactor 15 and a raw material solution which is separately prepared by allyl acetate may be supplied to the apparatus.

### EXAMPLES

Although the following provides a more detailed explanation of the present invention through examples thereof, the present invention is not limited thereto.

Analysis of each solution in Example and Comparative Examples were carried out by gas chromatography (GC). The evaluation conditions in the GC are shown below.

### [Gas Chromatography Analysis Conditions]

Instrument: GC-17A (Shimadzu Corp.)
Detector: Hydrogen flame ionization detector
Measurement method: Internal standard method (internal standard substance: 1,4-dioxane)
Injection temperature: 200° C.
Heating conditions: Held for 10 minutes at 40° C. followed by heating at 5° C./minute and holding for 30 minutes at 200° C.
Column used: TC-WAX (GL Science Inc., inner diameter: 0.25 mm, film thickness: 0.25 μm, and length: 30 m)

Reagents used are shown below.

Allyl acetate: marketed by Showa Denko K.K., purity: 99.6% by mass, impurity: 3594 ppm by mass of 1-propenyl acetate, 151 ppm by mass of acetic acid, and 59 ppm by mass of water

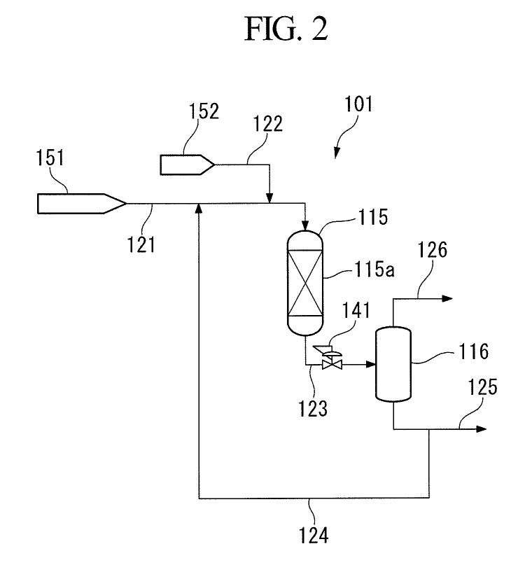

The first hydrogenation reaction apparatus, the second hydrogenation reaction apparatus, and the distillation apparatus used in this Example are shown in FIGS. 2 to 4.

As shown in FIG. 2, a first hydrogenation reaction apparatus 101 includes a first hydrogenation reactor 115 (a tubular reactor made of stainless having an inert diameter of 20 mm φ, inner volume: 80 cc), a gas-liquid separation tank 116, a flow path 121 for supplying the raw material solution containing allyl acetate 151, a flow path 122 for supplying the hydrogen containing gas 152, a flow path 123 having a secondary control valve 141 for supplying the hydrogenation reaction product from the first hydrogenation reactor 115 into the gas-liquid separation tank 116, a flow path 124 for supplying a part of the crude n-propyl acetate solution, which is the condensed component extracted from the gas-liquid separation tank 116, into the flow path 121 as the diluent solvent, a flow path 125 which is divided from the flow path 124 and extracts the crude n-propyl acetate solution from the gas-liquid separation tank 116, and a flow path 126 for extracting the non-condensed component from the gas-liquid separation tank 116.

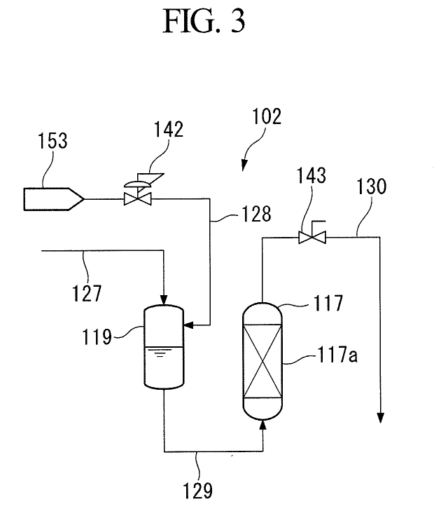

As shown in FIG. 3, the second hydrogenation reaction apparatus 102 includes a hydrogen dissolution tank 119 (volume: 100 cc) for further adding hydrogen in the crude n-propyl acetate solution produced in the first hydrogenation reaction apparatus 101, a second hydrogenation reactor (liquid phase reactor) 117 for hydrogenation of the non-reacted products contained in the crude n-propyl acetate solution produced in the first hydrogenation reaction apparatus 101, a flow path 127 for supplying the crude n-propyl acetate solution into the hydrogen dissolution tank 119, a flow path 128 having a secondary control valve 142 for supplying hydrogen 153 into the hydrogen dissolution tank 119, a flow path 129 for supplying the crude n-propyl acetate solution from the hydrogen dissolution tank 119 into the second hydrogenation reactor 117, and a flow path having a needle valve 143 for extracting the hydrogenation reaction solution from the second hydrogenation reactor 117.

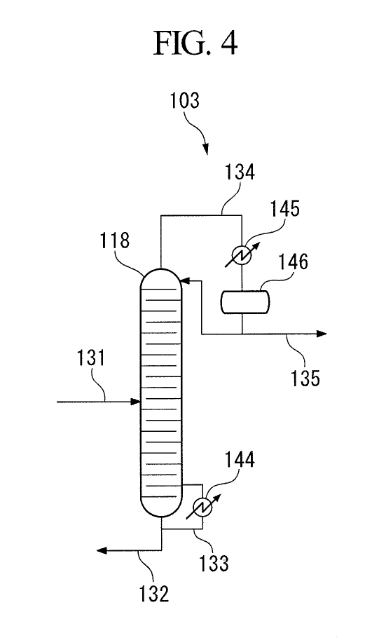

As shown in FIG. 4, the distillation purification apparatus 103 includes a distillation tower 118 (number of theoretical stages: 28), a flow path 131 for supplying the hydrogenation reaction solution produced in the second hydrogenation reaction apparatus 102 into the distillation tower 118, a flow path 132 for extracting the tower bottom solution from the bottom of the distillation tower 118, a flow path 133 which is divided from the flow path 132, and has a reboiler 144 for the tower bottom solution, a flow path 134 having a heat exchanger 145 and a condenser 146 for extracting and condensing the tower top solution from the top of the distillation tower 118 and returning the tower top solution into the distillation tower 118, and a flow path 135 which is divided from the flow path 134 and extracts 1-propenyl acetate.

### Example 1

As the hydrogenation catalyst, 80 cc of a support-type palladium catalyst (alumina support, pellets having 3 mm in diameter and 3 mm in length, content of palladium: 0.3% by mass, PGC catalyst marketed by N.E. Chemcat Corp.) was filled with the first hydrogenation reactor 115 of the first hydrogenation reaction apparatus 101 to produce a catalyst bed 115a. The inner pressure P₁ in the first hydrogenation reactor 115 was adjusted to 2.5 MPa G with hydrogen gas. Hydrogen gas was fed at the flow rate of 16.6 NL/hour into the first hydrogenation reactor 115 from the flow path 122. After the preset temperature in the electrical furnace in the first hydrogenation reactor 115 was adjusted to 80° C., a raw material solution was fed to the reactor (fixed-bed type, gas-liquid co-current down flow type reactor) at the flow rate of 400 g/hour from the top of the reactor. The raw material solution was prepared by mixing the recycle solution (the crude n-propyl acetate solution supplied from the flow path 124) and allyl acetate at a mass ratio of 9:1. The hydrogenation reaction product produced in the first hydrogenation reactor 115 was condensed in the gas-liquid separation tank 116. Then, in order to obtain the material balance in the reaction system, the crude n-propyl acetate solution was continuously extracted from the flow path 125. The obtained crude n-propyl acetate solution was analyzed with the GC.

The analysis results of the crude n-propyl acetate solution after reaching a steady state are shown below.
N-propyl acetate: 99.4464% by mass
Allyl acetate: 0.0493% by mass
1-propenyl acetate: 0.1479% by mass
Acetic acid: 0.3505% by mass
Water: 0.0058% by mass

The conversion of allyl acetate and selectivity of n-propyl acetate were calculated based on the analysis results as shown below.

X=1−(A+B)/(C+D)

Y=E/[(C+D)×X]

wherein X denotes the conversion (%) of allyl acetate, Y denotes the selectivity of n-propyl acetate, A denotes the amount (mole) of allyl acetate contained in the condensed component, B denotes the amount (mole) of 1-propenyl acetate contained in the condensed component, C denotes the amount (mole) of allyl acetate supplied from the flow path 121, D denotes the amount (mole) of 1-propenyl acetate contained in allyl acetate supplied from the flow path 121, and E denotes the amount (mole) of n-propyl acetate contained in the condensed component.

In the Example 1, X which is the conversion of allyl acetate in the first hydrogenation step was 99.8%, and Y which is the selectivity of n-propyl acetate was 99.43%.

A half of the hydrogen dissolution tank 119 in the second hydrogenation reaction apparatus 102 was filled with the crude n-propyl acetate solution produced in the first hydrogenation reaction apparatus 101. Then, hydrogen was further added in the crude n-propyl acetate solution from the flow path 128. At this time, the pressure in the hydrogen dissolution tank 119 was adjusted to 2.5 MPa G by the secondary pressure valve provided in the flow path 128. After that, the condensed component added with hydrogen was supplied into the second hydrogenation reactor 117 including a catalyst filling tank 117a filled with the 10 cc of the PGC catalyst. In the second hydrogenation reactor 117, the non-reacted products were hydrogenated in liquid phase at the pressure of P₂ (P₂: 2.5 MPa G). During the hydrogenation, the hydrogen dissolution tank 119, the flow path 129, and second hydrogenation reactor 117 were set in a water bath to control the temperature thereof.

The composition of the hydrogenation reaction solution obtained from the flow path 130 was analyzed with the GC by varying the control temperature, the detention time of the crude n-propyl acetate solution in the second hydrogenation reactor 117. The results are shown in Table 2. In Table 2, AAC and PEAC denote the allyl acetate and 1-propenyl acetate in the hydrogenation reaction solution obtained from the flow path 130, and the numbers in Table 2 are the concentration (ppm by mass) of AAC and PEAC.

TABLE 2
Unit: ppm by mass

| | | Reaction temperature | | | | | |
|---|---|---|---|---|---|---|---|
| | | 40° C. | | 60° C. | | 70° C. | |
| Kinds of component | | AAC | PEAC | AAC | PEAC | AAC | PEAC |
| Detention | 0 | 493 | 1479 | 493 | 1479 | 493 | 1479 |
| time | 1.7 | 20 | 30 | 15 | 26 | 12 | 25 |
| (min.) | 2.5 | 5 | 15 | 0 | 11 | 0 | 3 |
| | 5.0 | 0 | 13 | 0 | 8 | 0 | 2 |

As shown in Table 2, the concentration of allyl acetate and 1-propenyl acetate in the hydrogenation reaction solution is the lowest under condition in which the control temperature was 70° C. and the detention time was 5.0 minutes.

Then, distillation purification simulation was carried out in the distillation purification apparatus 103 using the hydrogenation reaction solutions in various conditions. In the distillation purification simulations, SimSci PRO/II v 8.1 was used. The hydrogenation reaction solution was supplied from the flow path 131 in the distillation tower 118 at the 21st stage from the top of the distillation tower 11. Moreover, the reflux ratio was set to 1, and the ratio (F/G) between the amount “F” of the extracted solution from the top of the distillation tower and the amount “G” of the solution supplied into the distillation tower was adjusted to 0.99.

Then, the composition of the solution supplied into the distillation tower (from the flow path 131), the product (n-propyl acetate, from the flow path 135), the distillation tower bottom solution (from the flow path 132), and the reflux solution (from the flow path 134) were analyzed with the GC. The results are shown in Table 3. In Table 3, allyl acetate and 1-propenyl acetate are assumed to be the same property, and the total amount thereof is denoted by the amount of allyl acetate.

TABLE 3

| | | | | Distillation | |
|---|---|---|---|---|---|
| | | | | tower | |
| | | Supplied | | bottom | Reflux |
| | | solution | Product | solution | solution |
| Temperature [° C.] | | 25 | 40 | 117.8 | 40 |
| Pressure [MPa G] | | 0.015 | 0 | 0.02 | 0 |
| Flow rate [kg/hour] | | 1000 | 990 | 10 | 990 |
| Composition | H₂O | 0.0058 | 0.0059 | 0 | 0.0059 |
| [% by mass] | allyl | 0.0002 | 0.0002 | 0.0001 | 0.0002 |
| | acetate | | | | |
| | n- | 99.6435 | 99.9939 | 64.9526 | 99.9939 |
| | propyl | | | | |
| | acetate | | | | |
| | acetic | 0.3505 | 0 | 35.0472 | 0 |
| | acid | | | | |

The amount of allyl acetate and 1-propenyl acetate could be reduced in Example 1 in which the pressure P₁ and P₂ in the first and second hydrogenation steps was adjusted to 2.5 MPa G. In addition, the purity of the product could be remarkably improved by the subsequent distillation operation, as shown in Table 3.

### Comparative Example 1

The distillation purification simulations using the distillation purification apparatus 103 was carried out in the same method as in Example 1, except that the crude n-propyl acetate solution produced in the first hydrogenation reaction apparatus 101 was used without the second hydrogenation step using the second hydrogenation reaction apparatus 102.

Then, the composition of the solution supplied into the tower (from the flow path 131), the product (n-propyl acetate, from the flow path 135), the distillation tower bottom solution (from the flow path 132), and the reflux solution (from the flow path 134) was analyzed with the GC. The results are shown in Table 4. In Table 4, allyl acetate and 1-propenyl acetate are assumed to be the same property, and the total amount thereof is denoted by the amount of allyl acetate, similar to in Table 3.

TABLE 4

| | | | | Distillation | |
|---|---|---|---|---|---|
| | | | | tower | |
| | | Supplied | | bottom | Reflux |
| | | solution | Product | solution | solution |
| Temperature [° C.] | | 25 | 40 | 117.8 | 40 |
| Pressure [MPa G] | | 0.015 | 0 | 0.02 | 0 |
| Flow rate [kg/hour] | | 1000 | 990 | 10 | 990 |
| Composition | H₂O | 0.0058 | 0.0059 | 0 | 0.0059 |
| [% by mass] | allyl | 0.1972 | 0.1979 | 0.1237 | 0.1979 |
| | acetate | | | | |
| | n- | 99.4465 | 99.7962 | 64.8291 | 99.7962 |
| | propyl | | | | |
| | acetate | | | | |
| | acetic | 0.3505 | 0 | 35.0472 | 0 |
| | acid | | | | |

It was impossible to sufficiently separate n-propyl acetate from allyl acetate and 1-propyl acetated by distillation in Comparative Example 1 in which the second hydrogenation step was not carried out. As shown in Table 4, the concentration of allyl acetate and 1-propenyl acetate in the product was high, and the product quality was inferior to that in Example 1.

### Comparative Example 2

The hydrogenation of allyl acetate was carried out in the same method as in Example 1, except that the pressure P₁ in the first hydrogenation reactor 115 was adjusted to 0.8 MPa G by hydrogen gas.

After reaching a steady state, the crude n-propyl acetate extracted from the flow path 125 was analyzed with the GC. The analysis results are shown below.
N-propyl acetate: 98.0931% by mass
Allyl acetate: 0.3200% by mass
1-propenyl acetate: 0.9599% by mass
Acetic acid: 0.6213% by mass
Water: 0.0058% by mass

Based on these results, X which is the conversion of allyl acetate in the first hydrogenation step was calculated 98.7%, and Y which is the selectivity percentage of n-propyl acetate was calculated 98.96% in the Comparative Example 2.

Then, the distillation purification simulations using the obtained crude n-propyl acetate were carried out with the distillation purification apparatus 103 in the same method as in Example 1.

Then, the composition of the solution supplied into the tower (from the flow path 131), the product (n-propyl acetate, from the flow path 135), the distillation tower bottom solution (from the flow path 132), and the reflux solution (from the flow path 134) was analyzed with the GC. The results are shown in Table 5. In Table 5, allyl acetate and 1-propenyl acetate are assumed to be the same property, and the total amount thereof is denoted by the amount of allyl acetate, similar to in Table 3.

TABLE 5

| | | | | Distillation | |
|---|---|---|---|---|---|
| | | | | tower | |
| | | Supplied | | bottom | Reflux |
| | | solution | Product | solution | solution |
| Temperature [° C.] | | 25 | 40 | 121.5 | 40 |
| Pressure [MPa G] | | 0.015 | 0 | 0.02 | 0 |
| Flow rate [kg/hour] | | 1000 | 990 | 10 | 990 |
| Composition | H₂O | 0.0058 | 0.0059 | 0 | 0.0059 |
| [% by mass] | allyl | 1.2799 | 1.2902 | 0.2508 | 1.2902 |
| | acetate | | | | |
| | n- | 98.093 | 98.7039 | 37.6241 | 98.7039 |
| | propyl | | | | |
| | acetate | | | | |
| | acetic | 0.6213 | 0 | 62.1251 | 0 |
| | acid | | | | |

The substrate conversion in the hydrogenation reaction at 0.8 MPa G was low and sufficiently separation of n-propyl acetate from allyl acetate and 1-propyl acetate was not carried out by distillation in Comparative Example 2 in which the pressure P₁ in the first hydrogenation step was adjusted to 0.8 MPa G by the addition of hydrogen gas and the second hydrogenation step was not carried out. As shown in Table 5, the concentration of allyl acetate and 1-propenyl acetate in the product was high, and the product quality was inferior to that in Example 1.

### Comparative Example 3

The second hydrogenation step was carried out in the same method as Example 1, except that the pressure in the hydrogen dissolution tank 119 and the pressure P₁ and P₂ in the first and second hydrogenation reactors 115 and 118 was adjusted to 0.8 MPa G.

Then, the composition of the hydrogenation reaction solution obtained from the flow path 130 was analyzed with the GC by varying the control temperature, the detention time of the crude n-propyl acetate in the second hydrogenation reactor 117. The results are shown in Table 6. In Table 6, AAC and PEAC denote the allyl acetate and 1-propenyl acetate in the hydrogenation reaction solution obtained from the flow path 130, and the numbers in Table 6 are the concentration (ppm by mass) of AAC and PEAC.

TABLE 6
Unit: ppm by mass

| | | Reaction temperature | | | | | |
|---|---|---|---|---|---|---|---|
| | | 40° C. | | 60° C. | | 70° C. | |
| Kinds of component | | AAC | PEAC | AAC | PEAC | AAC | PEAC |
| Detention | 1.7 | 2910 | 7610 | 2650 | 7310 | 2510 | 7230 |
| time | 2.5 | 2510 | 7130 | 2450 | 7020 | 2230 | 6960 |
| (min.) | 5.0 | 2420 | 7050 | 2210 | 6950 | 2050 | 6850 |

As shown in Table 6, the concentration of allyl acetate and 1-propenyl acetate in the hydrogenation reaction solution is the lowest under condition in which the control temperature was 70° C. and the detention time was 5.0 minutes.

Then, distillation purification simulations was carried out in the distillation purification apparatus 103 using a hydrogenation reaction solution obtained in various conditions in the same method as Example 1.

The composition of the solution supplied into the tower (from the flow path 131), the product (n-propyl acetate, from the flow path 135), the distillation tower bottom solution (from the flow path 132), and the reflux solution (from the flow path 134) was analyzed. The results are shown in Table 7. In Table 7, allyl acetate and 1-propenyl acetate are assumed to be the same property, and the total amount thereof is denoted by the amount of allyl acetate.

TABLE 7

| | | | | Distillation | |
|---|---|---|---|---|---|
| | | | | tower | |
| | | Supplied | | bottom | Reflux |
| | | solution | Product | solution | solution |
| Temperature [° C.] | | 25 | 40 | 121.5 | 40 |
| Pressure [MPa G] | | 0.015 | 0 | 0.02 | 0 |
| Flow rate [kg/hour] | | 1000 | 990 | 10 | 990 |
| Composition | H₂O | 0.0058 | 0.0059 | 0 | 0.0059 |
| [% by mass] | allyl | 0.8899 | 0.8971 | 0.1742 | 0.8971 |
| | acetate | | | | |
| | n- | 98.4831 | 99.0971 | 37.7108 | 99.0971 |
| | propyl | | | | |
| | acetate | | | | |
| | acetic | 0.6212 | 0 | 62.1151 | 0 |
| | acid | | | | |

The amount of allyl acetate and 1-propenyl acetate could not be sufficiently reduced in Comparative Example 3 in which the pressure P₁ and P₂ in the first and second hydrogenation steps was adjusted to 2.5 MPa G. In addition, allyl acetate and 1-propenyl acetate in the subsequent distillation operation could not sufficiently be removed. Due to this, the concentration of allyl acetate and 1-propenyl acetate in the product was high, and the product quality was inferior to that in Example 1, as shown in Table 7.

### INDUSTRIAL APPLICABILITY

The method for producing n-propyl acetate of the present invention is a method in which a hydrogenation reaction is performed with a hydrogenation catalyst using a raw material solution containing allyl acetate liquid. According to the production method of the present invention, it is possible to produce n-propyl acetate having a high quality, and prevent the product quality from deteriorating with time, which is caused by a decrease in the conversion rate of the substrate (allyl acetate).

[Explanation of reference symbols]

| | |
|---|---|
| 1: production apparatus for | 11: reactor |
| n-propylene acetate | |
| 12: condensation component | 13: oil-water separation tank |
| separation tank | |
| 14: first distillation tower | 15: first hydrogenation reactor |
| 16: gas-liquid separation tank | 17: second hydrogenation reactor |
| 18: second distillation tower | 18: raw material gas |
| 52: hydrogen containing gas | |
| 101: first hydrogenation reaction | |
| apparatus | |
| 102: second hydrogenation | 103: distillation purification apparatus |
| reaction apparatus | |
| 115: first hydrogenation reactor | 116: gas-liquid separation tank |
| 117: second hydrogenation reactor | 118: distillation tower |
| 119: hydrogen dissolution tank | |

1. A method for producing n-propyl acetate including:
a first hydrogenation step in which a raw material solution containing allyl acetate and a hydrogen containing gas are reacted under a pressure P₁ of 1.0 MPa G (gage pressure) or more in the presence of a hydrogenation catalyst, to hydrogenate the allyl acetate and produce a hydrogenation reaction product containing n-propyl acetate:
a gas-liquid separation step in which the hydrogenation reaction product is gas-liquid separated to produce a crude n-propyl acetate solution containing n-propyl acetate: and
a second hydrogenation step in which non-reacted allyl acetate contained in the crude n-propyl acetate solution is hydrogenated using hydrogen dissolved in the crude n-propyl acetate solution in the presence of a hydrogenation catalyst.

2. The method for producing n-propyl acetate according to claim 1, wherein a hydrogenation reaction in the second hydrogenation step is a liquid phase reaction.

3. The method for producing n-propyl acetate according to claim 1, wherein the pressure P₁ is in a range of 2.0 MPa G (gage pressure) to 20 MPa G (gage pressure).

4. The method for producing n-propyl acetate according to claim 1, wherein the ratio (P₂/P₁) between pressure P₂ in the second hydrogenation step and the P₁ is in a range of 0.9 to 2.0.

5. The method for producing n-propyl acetate according to claim 1, wherein the hydrogenation catalyst contains at least one metal selected from the group consisting of palladium, rhodium, ruthenium, nickel, and platinum.

6. The method for producing n-propyl acetate according to claim 1, wherein a reaction in the first hydrogenation step is a trickle bed type reaction.

7. The method for producing n-propyl acetate according to claim 1, wherein a molar ratio (Mₐ/M_{b}) between an amount (Mₐ mole) of supplied hydrogen and an amount (M_{b} mole) of supplied allyl acetate in the first hydrogenation step is in a range of 1.1 to 3.0.

8. The method for producing n-propyl acetate according to claim 1, wherein the pressure P₁ is in a range of 2.0 MPa G (gage pressure) to 20 MPa G (gage pressure).

9. The method for producing n-propyl acetate according to claim 2, wherein the ratio (P₂/P₁) between pressure P₂ in the second hydrogenation step and the P₁ is in a range of 0.9 to 2.0.

10. The method for producing n-propyl acetate according to claim 2, wherein the hydrogenation catalyst contains at least one metal selected from the group consisting of palladium, rhodium, ruthenium, nickel, and platinum.

11. The method for producing n-propyl acetate according to claim 2, wherein a reaction in the first hydrogenation step is a trickle bed type reaction.

12. The method for producing n-propyl acetate according to claim 2, wherein a molar ratio (Mₐ/M_{b}) between an amount (Mₐ mole) of supplied hydrogen and an amount (M_{b} mole) of supplied allyl acetate in the first hydrogenation step is in a range of 1.1 to 3.0.
